(19) United States
(12) Patent Application Publication
(10) Pub. No.: US 2005/0213710 A1
(43) Pub. Date: Sep. 29, 2005

(54) System and method for laser X-ray generation

(76) Inventors: Brian Lee Lawrence, Clifton Park, NY (US); Robert John Filkins, Niskayuna, NY (US)

Correspondence Address: Patrick S. Yoder;FLETCHER YODER, P.O. Box 692289, Houston, TX, 77269-2289, US

(21) Appl. No.: 10/812,211

(22) Filed: Mar. 29, 2004

(51) Int. Cl. G21K 1/12; H05G 1/60; A61B 6/00; G01N 23/00; H01J 35/00; H01J 35/22; H01J 35/32

(52) U.S. Cl. 378/121

(57) ABSTRACT

A technique is provided for using a laser based X-ray source for imaging systems such as computed tomography (CT) system. The laser based X-ray source may be provided as an X-ray bulb that emits X-rays when exposed to a laser beam. The X-ray bulb includes a bulb envelope and a bulb coating disposed on a portion of the bulb envelope, such that it acts as a focusing surface. The X-ray bulb also includes a target rotating about an axis such that a varying portion of the target passes through the focal point of the focusing surface. An X-ray producing plasma is generated when the laser beam is focused on the rotating target via the bulb coating.

### BACKGROUND

The present invention relates generally to X-ray sources, and in particular to a technique for generation of X-rays via a laser based X-ray source.

X-rays have found widespread applications in various medical and non-medical imaging techniques. For example, in a medical imaging technique known as computed tomography (CT) imaging, an X-ray source and a detector are mounted on a rotating gantry. As the CT gantry rotates around a patient, a fan or cone beam of X-ray passes through the patient body and the X-ray profile is created. Detectors measure this X-ray profile and produce electronic data pulses proportional to the intensity of the X-ray profile. These data pulses, from different detectors at different positions of the CT gantry, are fed into a computer, which uses them to form a digital image of the part of the patient through which the X-rays have passed.

In a typical CT imaging system and in other X-ray based imaging systems, an X-ray tube may be used to emit an X-ray beam directed towards the object to be imaged. In common X-ray tubes, Brehmsstrahlung radiation is produced when energetic electrons are decelerated by heavy materials. The X-rays produced in common X-ray tubes are generally of relatively low power, and comprise long pulses or a continuous wave that pose limitations in their use. Moreover, such radiation typically comprises fixed polarization, incoherent radiation that is not tunable. In addition, Brehmsstrahlung radiation requires highly energetic electron beams, which in turn require large and expensive facilities.

Current CT systems utilizing X-ray tubes are useful for three-dimensional imaging of human anatomy. However, the true utility of the CT process is restricted by the nature of the Brehmsstrahlung emission used to generate the images and the unwieldy mechanics of rotating a heavy and complex X-ray tube and detector plane array. The current CT systems may take longer than desired when scanning large organs or regions of a patient. This is inconvenient for the patient, especially children and those in emergency situations, as they have to hold their breath and stay motionless. In addition high gantry rotation speeds give better image resolution and offer the potential for stop-motion imaging of various organs, for example the heart.

One alternative to X-ray tubes, and their associated drawbacks, is to generate X-rays via a laser. In particular, high-powered lasers may be focused onto a target of appropriate material to generate plasma that subsequently produces X-rays. Alternatively, high-energy lasers interact with the accelerated electron beam to produce X-rays via the process of inverse Compton scattering. Laser based X-ray sources, due to their coherence and spectral properties offer significant benefits in lower dosage, higher-contrast, and better resolution over conventional X-ray tube technologies. However, current laser based X-ray sources are typically large and complex, and may not be easily implemented with existing imaging technologies, such as CT.

It is therefore desirable to provide a compact and effective X-ray source that is able to rotate about the patient at faster speeds and have narrow X-ray spectra so as to enable fast CT scanning with high resolution and at a lower doses thereby improving diagnosis and examination efficiency.

### BRIEF DESCRIPTION

Briefly in accordance with one aspect of the technique, an X-ray bulb is provided for generating X-rays when exposed to a laser beam. The X-ray bulb includes a bulb envelope and a bulb coating disposed on at least a part of a surface of the bulb envelope. The bulb coating is configured to form a focusing surface. The X-ray bulb also includes a target configured to rotate about an axis such that a varying portion of the target passes through the focal point of the focusing surface.

In accordance with another aspect of the technique, an imaging system is provided. The imaging system includes one or more X-ray bulb configured to emit X-rays at different locations relative to an imaging volume. Each X-ray bulb comprises a bulb envelope and a bulb coating disposed on at least a part of a surface of the bulb envelope. The bulb coating is configured to form a focusing surface. The X-ray bulb also includes a target configured to rotate about an axis such that a varying portion of the target passes through the focal point of the focusing surface. The imaging system also includes a laser source configured to generate a laser beam and a laser targeting system configured to focus the laser beam upon one of the bulb coatings at a time.

In accordance with a further aspect of the present technique, a method is provided for irradiating a volume. The method provides for moving an X-ray bulb relative to a volume to be imaged. The X-ray bulb may comprise a target configured to rotate about an axis such that a varying portion of the target passes through a focal point of a focusing surface formed by a bulb coating. In addition, the method provides for generating an X-ray producing plasma by focusing a laser onto the varying portion of the target via the bulb coating. Systems and computer programs that afford functionality of the type defined by this method may be provided by the present technique.

In accordance with an additional aspect of the present technique, a method is provided for irradiating a volume. The method provides for sequentially aiming a laser at each of a plurality of X-ray bulbs differentially positioned relative to a volume to be imaged. Each X-ray bulb comprises a target configured to rotate about an axis such that a varying portion of the target passes through a focal point of a focusing surface formed by a bulb coating. The method also provides for generating an X-ray producing plasma in each X-ray bulb by focusing the laser onto the varying portion of the respective target via the bulb coating when the laser is aimed at the respective X-ray bulb. Systems and computer programs that afford functionality of the type defined by this method may be provided by the present technique.

In accordance with another aspect of the present technique, a method is provided for generating X-rays. The method provides for rotating a target within an X-ray bulb and focusing a laser onto a focal point through which the target rotates. In addition, the method provides for indexing the target to raster the focal point such that the focal point successively focuses on a previously unexposed portion of the target. Systems and computer programs that afford functionality of the type defined by this method may be provided by the present technique.

### DRAWINGS

These and other features, aspects, and advantages of the present invention will become better understood when the following detailed description is read with reference to the accompanying drawings in which like characters represent like parts throughout the drawings, wherein:

FIG. 1 depicts an exemplary CT imaging system for volumetric imaging using an X-ray source in accordance with one aspect of the present technique;

FIG. 2 depicts a laser based X-ray generation technique in accordance with one aspect of the present technique;

FIG. 3 depicts an X-ray bulb for use in laser based X-ray generation in accordance with one aspect of the present technique;

FIG. 4 depicts an exemplary CT imaging system for volumetric imaging via an X-ray bulb of FIG. 3; and

FIG. 5 depicts an exemplary stationary CT imaging system for volumetric imaging via a plurality of X-ray bulbs of FIG. 3.

### DETAILED DESCRIPTION

The present techniques are generally directed to the generation of X-rays using lasers. Such laser based X-ray generation techniques may be useful in a variety of imaging contexts, including medical imaging and package and baggage screening. Though the present discussion provides examples in a medical imaging context, one of ordinary skill in the art will readily apprehend that the application of these techniques in non-medical contexts, such as for security screening, is well within the scope of the present techniques.

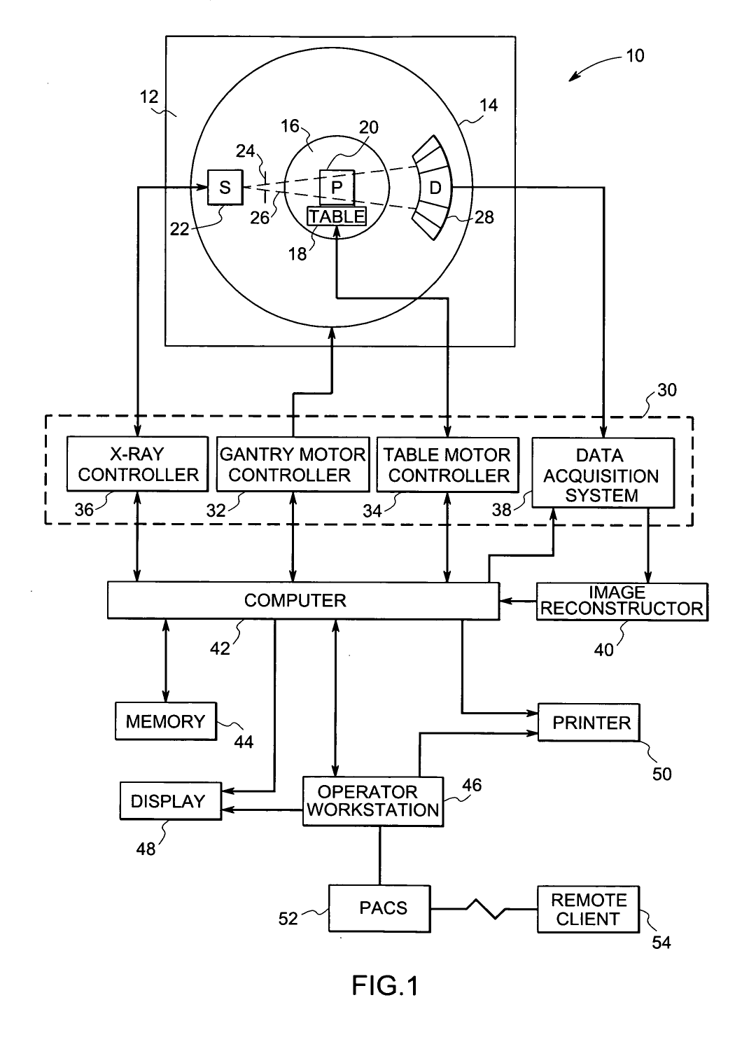

Referring now to FIG. 1, an imaging system 10 is illustrated for acquiring and processing image data. In the illustrated embodiment, the imaging system 10 is a computed tomography (CT) system designed both to acquire original image data and to process the image data for display and analysis in accordance with the present technique. The CT imaging system 10 is illustrated with a frame 12 and a gantry 14 that has an aperture (imaging volume or CT bore volume) 16. A patient table 18 is positioned in the aperture 16 of the frame 12 and the gantry 14. The patient table 18 is adapted so that a patient 20 may recline comfortably during the examination process.

The gantry 14 includes an X-ray source 22 positioned adjacent to a collimator 24. The collimator 24 typically defines the size and shape of the X-ray beam 26 that emerges from the X-ray source 22. In this exemplary embodiment, the X-ray source 22 is typically a laser based X-ray source in accordance with the present technique. In typical operation, the X-ray source 22 projects a stream of radiation (X-ray beam) 26 towards a detector array, represented generally at reference numeral 28, mounted on the opposite side of the gantry 14. Collimator 24 permits a stream of radiation 26 to pass into a particular region in which a subject, such as a human patient 20 is positioned. It should be noted that the particular region of the patient 20, for instance the liver, pancreas and so on, is typically chosen by an operator so that the most useful scan of a region may be acquired.

A portion of the radiation 26 passes through or around the subject and impacts the detector array 28. The detector array 28 may be a single slice detector or a multi-slice detector and is generally formed by a plurality of detection elements. Each detector element produces an electrical signal that represents the intensity of the incident X-ray beam 26 at the detector element when the beam 26 strikes the detector array 28. These signals are acquired and processed to reconstruct an image of the features within the subject 20.

Furthermore, the gantry 14 may be rotated around the subject 20 so that a plurality of radiographic views may be collected along an imaging trajectory described by the motion of the X-ray source 22 relative to the patient 20. In particular, as the X-ray source 22 and the detector array 28 rotate along with the CT gantry 14, the detector array 28 collects data of X-ray beam attenuation at the various view angles relative to the patient 20. Data collected from the detector array 28 then undergoes pre-processing and calibration to condition the data to represent the line integrals of the attenuation coefficients of the scanned objects 20. The processed data, commonly called projections, are then filtered and backprojected to formulate an image of the scanned area. Thus, an image or slice is acquired which may incorporate, in certain modes, less or more than 360 degrees of projection data, to formulate an image.

Rotation of the gantry 14 and operation of the source 22 is controlled by a system controller 30, which furnishes both power, and control signals for CT examination sequences. Moreover, the detector array 28 is coupled to the system controller 30, which commands acquisition of the signals generated in the detector array 28. The system controller 30 may also execute various signal processing and filtration functions, such as for initial adjustment of dynamic ranges, interleaving of digital image data, and so forth. In general, system controller 30 commands operation of the imaging system 10 to execute examination protocols and to process acquired data. In the present context, system controller 30 also includes signal processing circuitry, typically based upon a general purpose or application-specific digital computer, associated memory circuitry for storing programs and routines executed by the computer, as well as configuration parameters and image data, interface circuits, and so forth.

In the embodiment illustrated in FIG. 1, system controller 30 is coupled to the CT gantry 14 and patient table 18. In particular, the system controller 30 includes a gantry motor controller 32 that controls the rotational speed and position of the gantry 14 and a table motor controller 34 that controls the linear displacement of the motorized table 18 within the CT bore volume 16. In this manner, the gantry motor controller 32 rotates the CT gantry 14, thereby rotating the X-ray source 22, collimator 24 and the detector array 28 one or multiple turns around the patient 20. Similarly, the table motor controller 34 displaces the patient table 18, and thus the patient 20, linearly within the CT bore volume 16. Additionally, the X-ray source 22 may be controlled by an X-ray controller 36 disposed within the system controller 30. Particularly, the X-ray controller 36 may be configured to provide power and timing signals to the X-ray source 22.

Further, the system controller 30 may include a data acquisition system 38. In this exemplary embodiment, the detector array 28 is coupled to the system controller 30, and more particularly to the data acquisition system 38. The data acquisition system 38 typically receives sampled analog signals from the detector array 28 and converts the data to digital signals for subsequent processing. An image reconstructor 40 coupled to the computer 42 may receive sampled and digitized data from the data acquisition system 38 and performs high-speed image reconstruction. Alternatively, reconstruction of the image may be done by the computer 42. Once reconstructed, the image produced by the imaging system 10 reveals internal features of the patient 20.

The computer 42 is typically coupled to the system controller 30. The data collected by the data acquisition system 38 or the reconstructed images may be transmitted to the computer 42 and moreover, to a memory 44. It should be understood that any type of memory to store a large amount of data may be utilized by such an exemplary imaging system 10. Also the computer 42 may be configured to receive commands and scanning parameters from an operator via an operator workstation 46 typically equipped with a keyboard and other input devices. An operator may control the imaging system 10 via the operator workstation 46. Thus, the operator may observe the reconstructed image and other data relevant to the system from computer 42, initiate imaging, and so forth.

A display 48 coupled to the operator workstation 46 and the computer 42 may be utilized to observe the reconstructed image and to control imaging. Additionally, the scanned image may also be printed on to a printer 50 which may be coupled to the computer 42 and the operator workstation 46. Further, the operator workstation 46 may also be coupled to a picture archiving and communications system (PACS) 52. It should be noted that PACS 52 may be coupled to a remote system 54, such as radiology department information system (RIS), hospital information system (HIS) or to an internal or external network, so that others at different locations may gain access to the image and to the image data.

It should be further noted that the computer 42 and operator workstation 46 may be coupled to other output devices that may include standard or special purpose computer monitors and associated processing circuitry. One or more operator workstations 46 may be further linked in the imaging system 10 for outputting system parameters, requesting examinations, viewing images, and so forth. In general, displays, printers, workstations, and similar devices supplied within the imaging system 10 may be local to the data acquisition components, or may be remote from these components, such as elsewhere within an institution or hospital, or in an entirely different location, linked to the imaging system 10 via one or more configurable networks, such as the Internet, virtual private networks, and so forth.

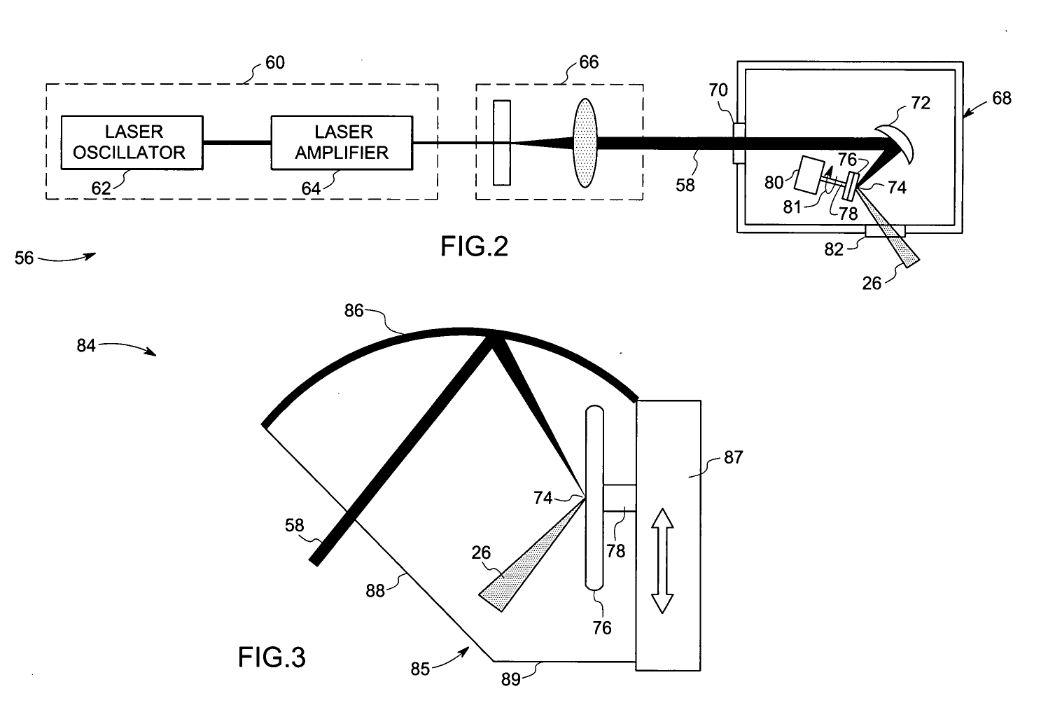

The exemplary imaging system 10, as well as other imaging systems based on X-ray attenuation, may employ X-ray sources 22 that generate X-rays 26 by a variety of techniques. For example, the present technique generates X-rays 26 using a laser. Referring now to FIG. 2, an exemplary embodiment 56 for generating X-rays 26 using a laser is depicted. In the depicted embodiment, a high-energy short-pulsed laser beam 58 is used to create a plasma that emits X-rays 26.

The laser beam 58 may be generated by a variety of techniques. For example, in the illustrated embodiment, a laser source 60 generates the high-energy laser beam 58. The laser source 60 typically includes a laser oscillator 62 producing a sub-picosecond laser beam. The output of the oscillator 62 is amplified via a laser amplifier 64 to deliver the high-energy laser beam 58.

In one implementation, a mode locked, diode pumped fiber laser is used as the laser oscillator 62. In such an implementation, the laser source 60 may include a CW, mode-locked laser oscillator that provides a train of 100 femtosecond (fsec) pulses, at a repetition rate of approximately 100 MHz. Each pulse may have approximately 10 nanojoules (nJ) of energy. The output of the oscillator 62 may be temporally stretched, regeneratively amplified, and recompressed in a laser amplifier 64 to provide 100 fsec pulses with in excess of 1 millijoule (mJ) of energy in each pulse at a repetition rate of less than 10 kHz or more specifically about 5 kHz. For example, the functions of the laser amplifier 64 may be performed by a chirped-pulse Ti:Al₂O₃ amplifier pumped by a Q-switched, lamp-pumped Nd:YAG laser. Alternatively a diode pumped amplifier may be used.

As will be appreciated by one of ordinary skill in the art, alternative implementations are also possible. For example, in one alternative implementation, a compact laser source 60 may be employed which provides a higher output energy. In such an implementation, the laser source 60 may include a diode-pumped Yb-doped mode-locked fiber laser as the laser oscillator 62 along with a diode-pumped Yb:YAG chirped-pulse laser amplifier. This type of laser source 60 is capable of more than 1 mJ (possibly as much as 5 mJ) of energy per pulse in a 100 fsec pulse at 5 kHz. In addition, as the system is diode pumped, the reliability, maintenance requirements, and lifetime is improved thereby reducing the associated costs.

As illustrated in FIG. 2, the laser beam 58 from the laser source 60 may pass through an expanding telescope 66, adjusted to give a parallel beam that may be directed into an X-ray source unit 68 through a laser transparent window 70. A focusing mirror 72 disposed in the X-ray source unit 68 directs the laser beam 58 towards its focal point 74.

A target 76 disposed in the X-ray source unit 68 is rotated about its axis via a shaft 78 coupled to a motor 80 in a direction 81 such that a varying portion of the target 76 passes through the focal point 74 of the focusing mirror 72. The target 76 is rotated so that each laser pulse is incident on previously unexposed material. Generally the target 76 is a metal or a metal alloy in the shape of a disk. For example, the target 76 may be composed of a high-Z metal with an atomic number greater than or equal to 40, such as tungsten, tantalum, bismuth, or others, which provides the desired X-ray spectrum and characteristic radiation. For example, a bismuth target might be used for characteristic radiation, K-alpha, of 78 KeV.

X-ray radiation may be generated when the laser beam 58 is focused onto the target 76, generating a plasma that emits X-rays 26. The emitted X-rays 26 exits the X-ray source unit 68 through an X-ray transmitting window 82 which may be made of beryllium or other X-ray transparent materials. As will be appreciated by those of ordinary skill in the art, the interior of the X-ray source unit 68 may be held in vacuum or alternatively, may contain a partial atmosphere of inert gas to control the re-deposition of the material ablated from the target 76.

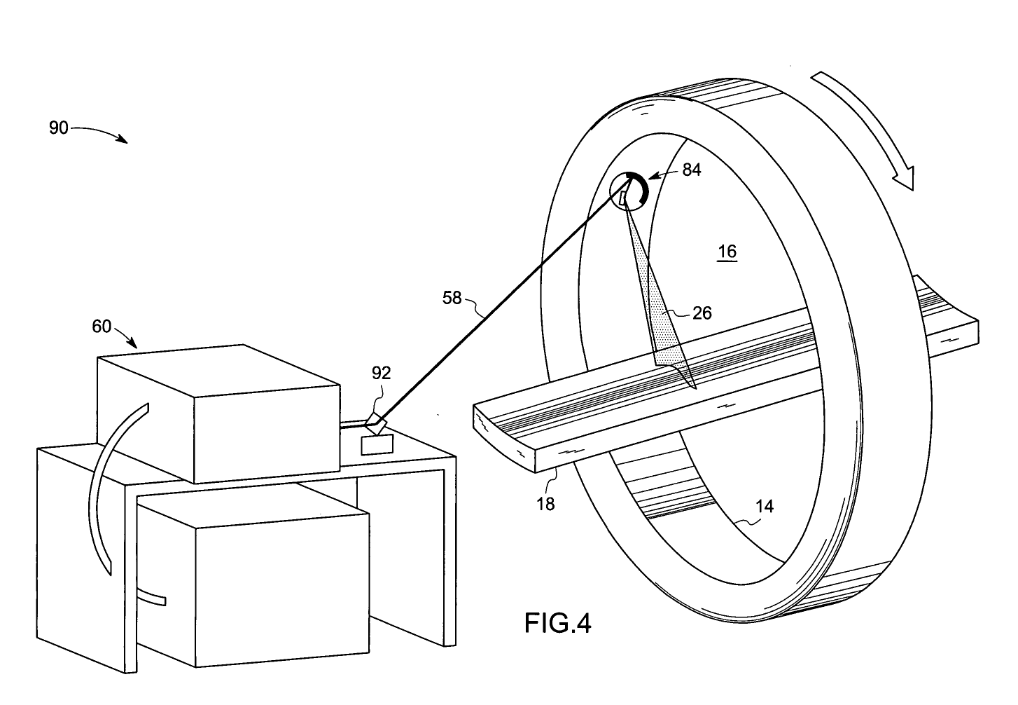

According to one aspect of the present technique, the X-ray source unit 68 of FIG. 2 may be provided as an X-ray bulb 84, as shown in FIG. 3. In one implementation of the X-ray bulb 84, the rotating target assembly (the target 76 and shaft 78 of FIG. 2) may be enclosed in a glass or a metal envelope 85. In one embodiment, a motor 80, as depicted in FIG. 2, may be located external to the X-ray bulb 84 such that it provides motive torque to a shaft 78 passing through the bulb envelope 85. In this implementation, the motor 80 may be a dedicated motor. Alternatively, the rotation of the target 76 may be coupled or geared to correspond to the translation of the X-ray bulb 84 relative to an object being imaged, such that at each location where X-rays 26 are to be generated the target 76 is rotated to present a previously unexposed portion of the target 76 to be impacted by the laser 58. For example, in rotational CT systems, the rotation of the target 76 may be driven by the revolution of the gantry about the image volume so that translating the X-ray bulb 84 to the different view angles rotates the target 76.

The bulb envelope 85 may be of a suitable shape and may be made, at least partially, of a laser transparent material, such as a laser transparent polymer or glass. The interior of the X-ray bulb 84 may be held in vacuum or, as noted above, may contain a partial atmosphere (such as about 20 torr) of an inert gas.

A portion of the surface of the bulb envelope 85, typically the internal surface, may be coated with a laser reflective material, typically a metal, such as gold, or a dielectric material. The laser reflective material may form a focusing surface 86, such as a spherical mirror or lens (similar to focusing mirror 72 as shown in FIG. 2), with the rotating target 76 located at the focal point 74 of the focusing surface 86. In such an implementation, the radius of curvature of the focusing surface 86 controls the focal length of the focusing surface 86. For example, in one embodiment, the radius of curvature is 16 cm.

A collimated laser beam 58 incident on the focusing surface 86 may be focused onto the target 76 at the focal point 74 to produce the X-ray emitting plasma. The target 76 may be indexed relative to the incoming laser beam 58 to raster the focal point 74 radially along the rotating target 76 such that previously unexposed portions of the target 76 successively pass through the focal point 74 to be impacted by the laser beam 58. In this manner, the surface area of the target 76 may be efficiently utilized.

With reference to FIG. 3, one implementation of such an X-ray bulb 84 is depicted. In this exemplary implementation, the bulb envelope 85 may include a metal mount 87 to which the shaft 78 is attached. The shaft 78 may be mounted to slide along the metal mount 87, which may facilitate moving the target 76 relative to the focal point 74 so that previously unexposed portions of the target 76 are impacted by the laser 58. The focusing surface 86 may be provided as a mirror-coated glass arc, which may be attached at one end to the metal mount 87 and at another to a laser and X-ray transparent material, such as an anti-reflection coated glass flat 88. As depicted, a second glass flat 89 may be employed to complete the bulb envelope 85.

In one implementation, the focusing surface 86 has an associated focal length of approximately 8 cm. In such an implementation, if the incident beam, i.e., laser 58, is maintained at 1 cm diameter, the laser 58 and focusing surface 86 combine to form an NA of ⅛, i.e., 0.125. At 800 nm, an NA of 0.125 produces a beam waist of approximately 6 microns. A beam waist of 6 microns combined with a laser producing 5 mJ at 100 femtoseconds produces a peak intensity in excess of 50×10¹⁵ W/cm². Such a peak intensity is sufficient to produce X-ray photon energies in excess of 80 keV. As will be appreciated by those of ordinary skill in the art, other peak intensities and X-ray photon energies may be obtained by adjusting characteristics of the X-ray bulb 84 and/or the laser 58. For example, other shapes or configurations of the X-ray bulb 84 may be employed based on the desired characteristics of the X-rays 26 or on the configuration of the imaging system employing the X-ray bulb 84.

For example, the X-ray bulb 84 may be incorporated as part of an X-ray based imaging system, such as a CT system or tomosynthesis system for medical imaging or a baggage or package screening device for non-medical imaging. In particular, an X-ray bulb 84 may be employed as an X-ray source 22 in an imaging system where it is moved relative to the imaged object, such as in a third-generation CT system or standard tomosynthesis system. Alternatively, multiple X-ray bulbs 84 may be employed as an X-ray source 22 in an imaging system where the X-ray bulbs do not move relative to the imaged object but are differentially activated to acquire X-ray projections at the desired view angles, such as in a stationary CT or stationary tomosynthesis system.

For example, referring generally to FIG. 4, an exemplary third-generation CT scanning system 90 is depicted in which an X-ray bulb 84 rotates on the gantry 14 about the aperture 16. As the X-ray bulb 84 rotates, a laser beam 58 from the laser source 60 tracks the X-ray bulb 84, allowing an X-ray beam 26 to be generated around the aperture volume 16. A laser targeting system 92, which may include a set of master-slave galvanometers or a two axis galvanometer may be used to steer the laser beam 58, allowing the laser beam 58 to track the X-ray bulb 84. The action of the laser targeting system 92, in conjunction with the rastering and indexing of the target 76, allow each laser pulse to be directed to a different point on the target 76 as the X-ray bulb 84 revolves about the imaged volume.

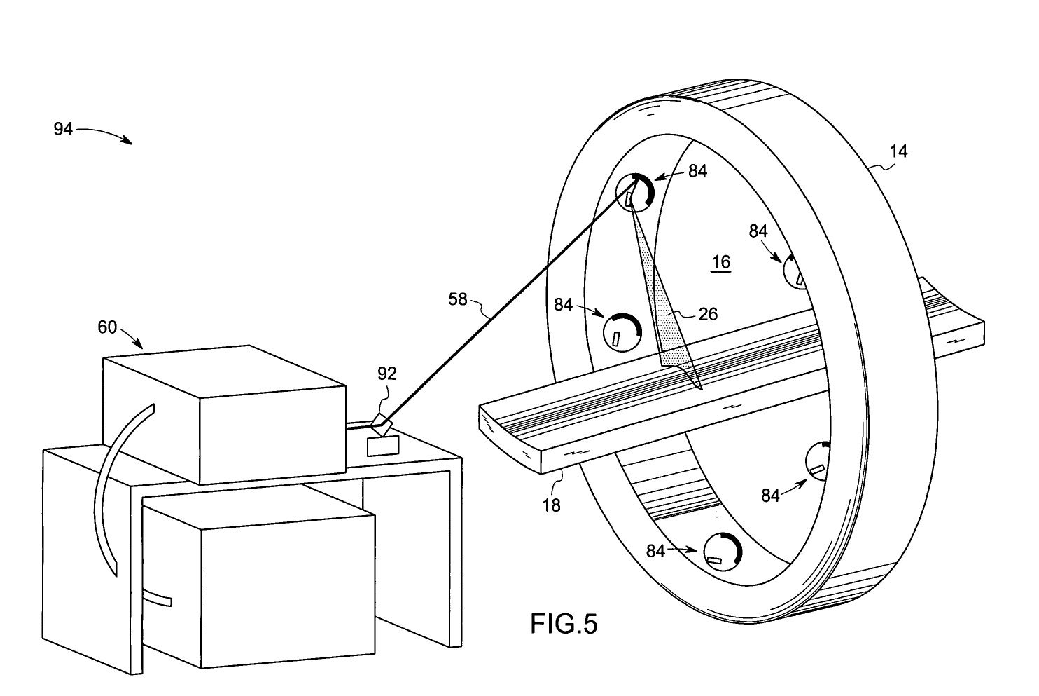

As noted above, imaging systems in which the X-ray source 22 is stationary may also be implemented using X-ray bulbs 84. For example, in a stationary CT system 94, as shown in FIG. 5, a plurality of X-ray bulbs 84 may be positioned at in a circle or arc about the aperture 16 so as to provide the desired angular coverage, such as 180° or 360°. In one implementation, the plurality of X-ray bulbs 84 may be positioned at regular intervals about the aperture 16. In other implementations, the X-ray bulbs 84 may be placed differentially, such that some portions of the aperture 16 have a differentially denser arrangement of X-ray bulbs 84, such as to accommodate differences in the path length through a patient 20. In stationary implementations, the laser targeting system 92 steers the laser beam 58 along the aperture 16 such that laser beam 58 hit the respective targets 76 associated with each X-ray bulb 84 at different times.

X-rays 26 generated via the laser based X-ray generation techniques as generally described above, may provide advantages over other X-ray generation techniques. For example, the laser X-ray generation techniques generally discussed herein may allow lower dosage, improved contrast, improved resolution, and/or new types of diagnostic imaging compared to other X-ray generation techniques, such as X-ray generation via conventional X-ray tubes. In particular, a laser focused to a small spot (i.e., focal point) produces effective X-ray source sizes of less than 50 microns, which may in turn improve image resolution. Furthermore, a stationary laser source 60 and small rotating X-ray bulb 84 may reduce the CT gantry structural requirements and may thereby allow for increased gantry rotation speeds. In addition, the rotating X-ray bulb may be configured as a purely passive device, with target rotation and indexing handled by a cradling mechanism on the CT gantry 14. Additionally, no cooling of the target is required. Furthermore, the replacement cost of an X-ray bulb 84, relative to a conventional X-ray tube, may be significantly less. In addition, the X-ray bulbs may be user replaceable, resulting in increased system availability and convenience. As a consequence, imaging systems employing X-ray bulbs may be easier to maintain and less expensive than imaging systems based on X-ray tubes.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An X-ray bulb comprising:
a bulb envelope;
a bulb coating disposed on at least a part of a surface of the bulb envelope, wherein the bulb coating is configured to form a focusing surface; and
a target configured to rotate about an axis such that a varying portion of the target passes through a focal point of the focusing surface.

2. The X-ray bulb of claim 1, wherein the axis is geared to rotate based upon the motion of the X-ray bulb about a volume to be imaged.

3. The X-ray bulb of claim 1, wherein the bulb coating is disposed on an interior surface of the bulb envelope.

4. The X-ray bulb of claim 1, wherein the target comprises at least one of a metal and a metal alloy.

5. The X-ray bulb of claim 1, wherein the target comprises a metal having an atomic number of at least about 40.

6. The X-ray bulb of claim 1, wherein the bulb envelope comprises glass.

7. The X-ray bulb of claim 1, wherein the bulb envelope comprises a laser transparent material.

8. The X-ray bulb of claim 1, wherein the bulb envelope comprises a laser transparent polymer.

9. The X-ray bulb of claim 1, wherein at least a partial atmosphere of an inert gas is within the bulb envelope.

10. The X-ray bulb of claim 1, wherein the bulb coating comprises at least one of a metal and a dielectric material.

11. An imaging system comprising:
one or more X-ray bulbs configured to emit X-rays at different locations relative to an imaging volume, each X-ray bulb comprising:
a bulb envelope;
a bulb coating disposed on at least a part of a surface of the bulb envelope, wherein the bulb coating is configured to form a focusing surface; and
a target configured to rotate about an axis such that a varying portion of the target passes through a focal point of the focusing surface;
a laser source configured to generate a laser beam; and
a laser targeting system configured to focus the laser beam upon one of the bulb coatings at a time.

12. The imaging system of claim 11, further comrpising a motion subsystem configured to move the one or more X-ray bulbs along an imaging trajectory.

13. The imaging system of claim 12, wherein the motion subsystem is configured to move the one or more X-ray bulbs along a tomosynthesis imaging trajectory.

14. The imaging system of claim 12, wherein the motion subsystem is configured to move the one or more X-ray bulbs by moving a CT gantry.

15. The imaging system of claim 11, wherein the imaging volume comprises a tomosynthesis imaging volume.

16. The imaging system of claim 11, wherein the imaging volume comprises a CT bore volume.

17. The imaging system of claim 11, wherein the one or more X-ray bulbs comprise a plurality of X-ray bulbs positioned generally around at least a portion of the imaging volume.

18. The imaging system of claim 11, wherein the one or more X-ray bulbs comprise a plurality of X-ray bulbs positioned at substantially equal intervals about the imaging volume.

19. The imaging system of claim 11, further comprising one or more detector arrays disposed about the imaging volume such that X-rays emitted by the one or more X-ray bulbs impact the one or more detector arrays.

20. The imaging system of claim 11, wherein a respective axis is geared to rotate based upon the motion of the respective X-ray bulb about the imaging volume.

21. The imaging system of claim 11, wherein the laser targeting system comprises a two-axis galvanometer.

22. The imaging system of claim 11, wherein the laser source comprises at least a laser oscillator and a laser amplifier.

23. A method for irradiating a volume, the method comprising:
moving an X-ray bulb relative to a volume to be imaged, the X-ray bulb comprising a target configured to rotate about an axis such that a varying portion of the target passes through a focal point of a focusing surface formed by a bulb coating; and
generating an X-ray producing plasma by focusing a laser beam onto the varying portion of the target via the bulb coating.

24. The method of claim 23, wherein moving the X-ray bulb comprises rotating a CT gantry to which the X-ray bulb is attached.

25. The method of claim 23, wherein moving the X-ray bulb comprises moving the X-ray bulb along a tomosynthesis imaging trajectory.

26. The method of claim 23, further comprising detecting the X-rays on one or more detector arrays.

27. The method of claim 23, further comprising generating one or more projection images based upon signals produced by the one or more detector arrays in response to the detected X-rays.

28. A method for irradiating a volume, the method comprising:
sequentially aiming a laser beam at each of a plurality of X-ray bulbs differentially positioned relative to a volume to be imaged, wherein each X-ray bulb comprises a target configured to rotate about an axis such that a varying portion of the target passes through a focal point of a focusing surface formed by a bulb coating; and
generating an X-ray producing plasma in each X-ray bulb by focusing the laser beam onto the varying portion of the respective target via the bulb coating when the laser beam is aimed at the respective X-ray bulb.

29. The method of claim 28, wherein the volume to be imaged comprises a tomosynthesis imaging volume.

30. The method of claim 28, wherein the volume to be imaged comprises a CT bore volume.

31. The method of claim 28, wherein the plurality of X-ray bulbs are positioned generally around at least a portion of the volume to be imaged.

32. The method of claim 28, wherein the plurality of X-ray bulbs are positioned at substantially equal intervals about the volume to be imaged.

33. The method of claim 28, further comprising detecting the X-rays on one or more detector arrays.

34. The method of claim 33, further comprising generating one or more projection images based upon signals produced by the one or more detector arrays in response to the detected X-rays.

35. A method for generating X-rays, the method comprising:
rotating a target within an X-ray bulb;
focusing a laser beam onto a focal point through which the target rotates; and
indexing the target to raster the focal point such that the focal point successively focuses on a previously unexposed portion of the target.

36. The method of claim 35, wherein focusing the laser beam comprises focusing the laser beam upon an interior bulb coating which focuses the laser beam onto the focal point.

37. The method of claim 35, wherein the focal point is rastered radially along the target.

38. The method of claim 35, further comprising moving the X-ray bulb about a volume to be imaged.
